(19) United States
(12) Patent Application Publication
(10) Pub. No.: US 2005/0152986 A1
(43) Pub. Date: Jul. 14, 2005

(54) Cross-linked collagenous biomaterial

(76) Inventors: Nicolaas Duneas, Boksburg North (ZA); Martina Magdel Lutz, Johannesburg (ZA)

Correspondence Address: BRINKS HOFER GILSON & LIONE, P.O. BOX 10395, CHICAGO, IL, 60610, US

(21) Appl. No.: 10/489,084

(22) PCT Filed: Sep. 4, 2002
(86) PCT No.: PCT/IB02/03576

(30) Foreign Application Priority Data
Sep. 6, 2001 (ZA) 2001/7385

(51) Int. Cl. A61K 35/32

(52) U.S. Cl. 424/549

(57) ABSTRACT

A method or producing a cross-linked collagenous biomaterial includes the step of providing a collagenous biomaterial and irradiating the collagenous biomaterial with gamma radiation at a dose of between about 20 and 160 kGy to produce the cross-linked collagenous biomaterial. The collagenous biomaterial is provided in the form of a gel and is produced by extracting bone powder or tendon.

THIS INVENTION relates to collagenous biomaterials.

More particularly, the invention relates to a method of producing a cross-linked collagenous biomaterial, to a cross-linked collagenous biomaterial produced by the method, to a tissue-regenerating composition, to a tissue-regenerating device, to the use of a substance or composition in the preparation of a medicament for the regeneration of tissue, to a method of regenerating tissue and to a substance or composition for use in a method of regenerating tissue.

The cross-linked collagenous biomaterials of the invention are particularly intended for human tissue regeneration and treatment and for promoting or inducing bone growth.

According to a first aspect of the invention, there is provided a method of producing a cross-linked collagenous biomaterial, the method including the steps of providing a collagenous biomaterial and Irradiating the collagenous biomaterial with gamma radiation at a dose of between about 20 and 160 kGy to produce the cross-linked collagenous biomaterial.

The collagenous biomaterial may be provided in the form of a gel. The collagenous biomaterial may be irradiated at a dosage of between about 70 and 90 kGy and preferably at a dosage of about 80 kGy. The method may thus include the step of irradiating the gel to form the cross-linked collagenous biomaterial.

The collagenous biomaterial used in the cross-linking step of the invention may be a mixture or extract containing collagen. It may be produced by extracting bone powder. It may, instead, be produced by extracting tendon. The bone powder may be defatted, dehydrated, milled human cortical bone powder.

For example, undemineralised bone powder may be boiled in water to produce an extract which is rich in collagen type I. Instead, a mixture of collagen and elastin may be extracted from human tendon by boiling the tendon in an aqueous acidic medium. These extracts contain minerals and other proteins which are co-extracted with the collagen. Typically, the material comprises collagen type 1 and other proteins native in bone powder. The collagenous material typically contains collagen type 1 in high abundance, lesser quantities of other native proteins, trace levels of minerals (solubilised from bone during the extraction step) and trace quantities of sugars and carbohydrates.

The extraction step may involve extracting the bone powder with hot water or extracting the tendon with hot aqueous acid e.g. hot aqueous acetic acid to produce an aqueous extract.

The extraction step may thus be selected from an aqueous extraction step and an aqueous acidic extraction step.

The method may include the step of isolating the collagenous biomaterial, dissolving the collagenous biomaterial in a pre-determined volume of water to produce a solution of the biomaterial and allowing the solution to set to form a gel.

Isolating the collagenous biomaterial may be by evaporating the aqueous extract or by precipitation of the collagenous biomaterial using a water soluble solvent such as ethanol. The aqueous extract may be concentrated in a microwave heating step.

The gel may be irradiated with a cobalt 60 source, for example by using a Source Nordion C-188 irradiator.

The invention extends to a cross-linked collagenous biomaterial prepared by a method as hereinbefore described.

The cross-linked material may be further processed in the wet state by mincing it to form a paste-like material and admixing the paste like material with substances selected from processed tissue banked bone, insoluble collagenous bone matrix, bone growth inducing factors, cortical or cancellous bone particles, sintered and powdered hydroxyapatites, ceramic powders, demineralised bone particles and mixtures thereof to produce novel delivery systems for tissue regeneration.

The invention thus extends to a tissue-regenerating composition which includes a cross-linked collagenous biomaterial produced by a method as hereinbefore described and at least one component selected from processed bone, insoluble collagenous bone matrix, bone growth inducing factors, cortical bone particles, cancellous bone particles, hydroxyapatites, ceramic powders, demineralised bone particles and mixtures of any two or more thereof.

The bone growth inducing factors may be selected from bone morphogenetic proteins, transforming growth factor beta and combinations thereof.

The collagenous material may instead be freeze-dried and milled to a known particle size range, and packaged in a syringe either alone or as a composition with one or more of the substances referred to above which may be rehydrated to form an injectable device useful for the treatment of bone defects in a human or animal.

The invention thus extends further to a tissue-regenerating device which includes a tissue-regenerating composition as hereinbefore described and administration means for administering the composition to a treatment site. The administration means may be a syringe.

Instead, the administration means may be in the form of a carrier such as a membrane, sponge or sheet. For example, it may be in the form of a membrane for use in guided tissue regeneration such as periodontal regeneration, or in the form of a haemostatic sponge for use in stopping haemorrhagic trauma of internal organs such as the liver or spleen or in the form of a sheet for use in covering skin burns.

The phrase “collagenous bone matrix” refers to material which is dissociatively extracted with chaotropic agents such as strong urea or guanidinium solutions from milled demineralised bone powder. The phrase “bone morphogenetic proteins” refers broadly to protein morphogens which are known to induce bone formation in primates when delivered to recipient skeletal sites in conjunction with a suitable carrier material. The phrase “type I collagen” refers to purified preparations of type I collagen which are more than 98% pure.

According to another aspect of the invention there is provided the use of a substance or composition in the preparation of a medicament for the regeneration of tissue, the substance or composition including a cross-linked collagenous biomaterial prepared by a method as hereinbefore described.

The substance or composition may include at least one component selected from processed bone, insoluble collagenous bone matrix, bone growth inducing factors, cortical bone particles, cancellous bone particles, hydroxyapatites, ceramic powders, demineralised bone particles and mixtures of any two or more thereof.

According to another aspect of the invention there is provided a method of regenerating tissue, the method including the step of administering to a person or animal in need of treatment an effective amount of a substance or composition which includes a cross-linked collagenous biomaterial prepared by a method as hereinbefore described.

The substance or composition may include at least one component selected from processed tissue banked bone, insoluble collagenous bone matrix, bone growth inducing factors, bone differentiating factors, cortical or cancellous bone particles, sintered and powdered hydroxyapatites, demineralised bone particles, ceramic powders and mixtures thereof.

Bone differentiating factors comprise a large family of proteins which function during embryo formation to cause morphogenesis. This is the formation or construction of tissue with form and function and which take on their respective role in the body. Examples are tissues of bone, cartilage, skin, liver, brain and lung. Some of the growth and differentiating factors are redeployed in adults to cause regeneration of tissue via mechanisms closely resembling embryonic differentiation. Examples of these factors are the bone morphogenetic proteins which induce new bone formation when implanted in an adult.

The method may include treating human bone defects by local application of the medicament directly to bone defects, either by implantation or by injection, for example in humans suffering from bone loss resulting from trauma, tumour resection, osteoporosis, tooth extraction, radiation damage or infectious disease such as tuberculosis or periodontitis.

According to another aspect of the invention, there is provided a substance or composition for use in a method of regenerating tissue, the substance or composition comprising a cross-linked collagenous biomaterial prepared by a method as hereinbefore described and the method comprising administering to a person or animal in need of treatment an effective amount of the substance or composition.

The substance or composition may include at least one component selected from processed tissue banked bone, insoluble collagenous bone matrix, bone growth inducing factors, cortical or cancellous bone particles, sintered and powdered, hydroxyapatites, demineralised bone particles, ceramic powders and mixtures thereof. The bone growth inducing factors may be as hereinbefore described.

The cross-linking of collagen has been widely described in the literature as a method useful for improving the physical properties and biocompatibility of collagen. Prior art cross-linking has generally involved reacting collagen with a cross-linking agent such as dimethyl-3,3′-dithiobispropionimidate (Charulatha V, Rajeram A J(2001) Dimethyl-3,3′-dithiobispropionimidate: a novel cross-linking reagent for collagen; Biomedical Materials Research; January; 54(1):122-8) or an aldehyde such as gluteraldehyde or formaldehyde. However, a disadvantage of using chemical cross-linking agents is the cytotoxic nature of these compounds. Collagen is a protein and the chemical cross-linking agents used to cross-link collagen have the capacity also to attack other proteins in the body. A requirement for the manufacture of safe collagenous materials for human use is accordingly the removal of residual cross-linking agent from the cross-linked collagen after the cross-linking step.

It is an object of the present invention to provide a novel method for the preparation of cross-linked collagenous biomaterials with useful biological and physical properties.

It has previously been found that collagen molecules are readily damaged by gamma-radiation at dosages commonly used for sterilizing biomedical products. It has been found that irradiating collagen or chemically cross-linked collagen at a dose which is higher than about 10 kGy causes significant damage to both the collagen and the cross-linked collagen. A significant number of peptide bonds are cleaved by the irradiation and this causes considerable changes in the characteristics of the material. It has been shown (Cheung D T, Perelman N, Tong O, Nimni M E J (1990) The effect of gamma-irradiation on collagen molecules, isolated alpha-chains, and crosslinked native fibers in Biomed Mater Res May 1990; 24(5):581-9.) that the irradiation of purified type I collagen from collagenous bone matrix results in radiation damage to the extent that the majority of the gel strength and a sticky properties are lost.

The invention now provides a novel method for cross-linking collagen without using a cross-linking agent. The invention involves the isolation of a collagenous material which is rich in collagen type I from human tissue banked bone or human bone tendon, the preparation of a gel with a specific range of protein concentration from the isolated collagenous material and the exposure of the gel to gamma irradiation to cause cross-linking. The gel has been found to have a markedly better gel strength and stickiness compared to material prepared from highly purified human type I collagen (98% pure) using prior art methods.

The invention is now described, by way of example, with reference to the accompanying Examples and Tables.

### EXAMPLE 1

Diaphyseal human long bone shafts were cut longitudinally, demarrowed, and cleaned of adhering soft tissues until only the cortical bone remained. In another embodiment of the invention the bone shafts were bovine bone shafts. The cut pieces were reduced in size to small pieces and defatted with 3 to 5 volumes of ethyl ether, dehydrated with 3 volumes of ethyl alcohol and air dried. In another embodiment of the invention the pieces were defatted with a 1:1 mixture of chloroform and methanol. The defatted dried material was milled to a particle size of less than 75 microns and boiled in five volumes of purified water in a pressure vessel for one to two hours. In another embodiment of the invention, the dried material was boiled in physiological saline solution. In still another embodiment of the invention, the dried material was boiled in an open vessel ensuring replenishment of water lost by evaporation.

The resulting suspension was filtered using a paper filter and the resulting clarified liquid was concentrated by evaporation on a hot plate to produce the collagenous biomaterial. In another embodiment of the invention the collagenous biomaterial was precipitated from solution by the addition of three to four volumes of chilled absolute ethyl alcohol, dried under vacuum and weighed. In another embodiment of the invention, the collagenous biomaterial was concentrated by boiling in a microwave oven, dried under vacuum and weighed.

An aqueous solution of this material at a concentration of about 15% mass by volume was made up by dissolving the appropriate amount of material in hot water above 80 degrees Celsius until the material had fully dissolved and a clear, pale yellow solution had been produced. In other embodiments of the invention, the concentration of the aqueous solution was between 5% and 20%. The clear solution was decanted into smaller volumes and allowed to set to a solid gel. In different embodiments of the invention the clear solution was allowed to set at room temperature and in a refrigerator at 4 degrees Celsius.

The gel was packaged, sealed and irradiated at 80 kGy using a cobalt 60 source from a Sourca Nordion C-188 irradiator to cause cross-linking. In different embodiments, the gel was irradiated at doses which varied between 20 and 160 kGy. The irradiation was carried out at room temperature at ambient conditions of temperature and humidity.

The cross-linked gel was then wet-milled using a blending device which comprises a holding vessel equipped with blades mounted on a rotating drive shaft. Blending was stopped when a fine gelatinous and highly viscous material had been obtained. In different embodiments of the invention, this material was combined with bone chips, DFDBA (defatted, demineralised, freeze-dried bone allograft), collagenous bone matrix or active growth factors to produce compositions. The compositions were packed into syringes and sterilized further by standard doses of gamma irradiation of 2.5 kGy at a temperature of −40 to −20 C. These biological devices were successfully used in sinus lift procedures in humans to regenerate and augment bone in the sinus region for the purposes of obtaining sufficient bone depth in which to place implants intended for tooth prosthesis.

Tables 1A and 1B set out the results of tests with a composition comprising cross-linked collagen together with cortical or cancellous demineralised or undemineralised bone particles in the promotion of bone formation in human sinus lift procedures. Maxillary sinus bone changes are shown in mm of new bone generated after 5 and 3 months as judged from before and after radiographs of humans treated with the composition described in Table 1A.

TABLE 1A

| MATERIAL | AMOUNT |
|---|---|
| Milled 16% strength cross-linked | 60% |
| collagen gel | |
| Cortical human bone chips | 40% |

TABLE 1B

| | Trial | Amount of | Amount of bone |
|---|---|---|---|
| | period | medicament | generated |
| Case number | (months) | used (ml) | (mm) |
| 1 | 5 | 2 | 9 |
| 2 | 3 | 2 | 8 |

A composition comprising cross-linked collagen together with bone morphogenetic proteins and collagenous bone matrix was found to induce new bone formation when injected into soft tissues of the rodent and bony sites of the human. Results are set out in Table 2.

TABLE 2

| | AMOUNT |
|---|---|
| MATERIAL | milligrams |
| Milled 16% strength cross-linked | 1000 |
| collagen gel | |
| Human demineralised bone matrix | 500 |
| Human bone morphogenetic protein | 0.5-2.5 |

The same material may be injected locally into osteoporotic lesions of the human skeleton to assist in bone regeneration where bone loss has occurred. A composition comprising cross-linked collagen together with apatite powders has been found to be a useful bone filling material.

### EXAMPLE 2

Human achilles tendon was wet milled in 10 volumes of purified water containing 0.2 N acetic acid and autoclaved for 20 minutes in a glass bottle. The supernatant was filtered off and precipitated with 4 volumes of chilled absolute alcohol. The precipitated material was dried in vacuo and reconstituted with purified water at 80 degrees Celsius. In different embodiments of the invention, the amount of water used to reconstitute the precipitated material was varied to produce a concentration of between 2% and 20%. The material was poured into moulds and allowed to set to a gel at 4 degrees Celsius. The gel was then irradiated with a cobalt 60 source at a dosage of at 80 kGy to effect cross-linking. In different embodiments of the invention, the gel was irradiated at dosages which varied between 20 and 160 kGy The material was then wet milled and combined with the biological materials as described in Example 1.

### EXAMPLE 3

Processing Protocoles for Syringed Cross-Linked Collagen Bone

Diaphyseal human long bone shafts were cut longitudinally, demarrowed, and cleaned of soft tissues until only the cortical bone remained. The cut strips were further reduced in size to about 15 mm×15 mm and defatted in three volumes of a 1:1 mixture of chloroform and methanol for 24 hours at room temperature. The solvents were discarded and the bone was defatted in a fresh solution of chloroform and methanol for a further 24 hours.

The defatted bone was then dehydrated with 3 volumes of ethyl alcohol for 24 hours and air dried. The defatted, air-dried material was milled to a particle size of less than 125 micron.

The fine bone powder was then boiled in Schott high-pressure glass vessels in five volumes of distilled water in a pressure cooker at near max temperature for a minimum 2 hours. After partial cooling, the bottles were vigorously shaken to further separate the mineral and collagenous components, the lids were unscrewed and the mixture was left to settle into two separate components.

The yellow collagenous material was then drawn off with a syringe and filtered through a paper filter. The filtrate was boiled in a vessel in a microwave oven for several minutes on maximum output, to remove about 75% of the water. A perforated paper filter was used to cover the vessel to prevent loss of material caused by sputtering during the microwave step. A dark yellow, highly viscous concentrated collagen material was obtained.

This material was gelled at room temperature in 30 cc clear, polyethylene screw-cap containers and then subjected to 80 kGy crosslinking at ambient temperature and humidity. The jars were labelled with the date, dosage and batch number. Irradiation was conducted with a Nordion C-188 Cobalt 60 source.

The cross-linked product was then wet milled with a blending device comprising a holding vessel equipped with blades mounted on a rotating drive shaft made by Braun, deep frozen to −80 degrees Celsius and lyophilized for three days to complete dryness. The dry, lyophilized product was milled in an IKA milling machine fitted with a 1 mm mesh.

This material was used to prepare injection devices in accordance with the invention. In each case, the cross-linked collagen was dissolved in purified water BP to produce a 20% mlv mixture and rehydrated at 4 Degrees Celsius overnight. The devices are referred to as
COB-10, COB-05, COB-02 and COB-01,
CAB-02 and CAB-01, and
CCAB-0.5, CCAB-02 and CCAB-01

The ratio of collagen to DFDBA in COB-10, COB-05, COB-02, COB-01, CAB-02 and CAB-01 was 60:40 and the ratio of collagen to DFDBA in CCAB-0,5, CCAB-02 and CCAB-01 was 40:60. DFDBA refers to demineralised, freeze-dried bone allograft.

The DFDBA particles were sifted very thoroughly to obtain material having a particle size between 250 and 1000, micron. Madison test sieves were used to obtain this particle size by sifting the material three times and discarding any material outside these parameters.

COB-10

The cross-linked collagen (60 g) was measured out and wet milled again using the blending device described above, The sifted DFDBA particles were poured into a graded measuring device and compacted lightly by tapping five times on a hard surface.

A compacted volume of 40 cc cortical DFDBA was added to the measured collagen and mixed thoroughly, first with the blending device and then by extrusion from one Promex 50 cc catheter tip syringe to another. A completely smooth and homogeneous mixture was obtained.

This material was then loaded into Becton-Dickinson 10 cc syringes by removing the plunger and extruding 10 cc into the syringe. The plunger was replaced and air removed from the system. The nozzle was then sealed with a tight-fitting cap. The B-D syringes all had nozzle apertures that had been enlarged to 3 mm. The syringes were then packed in two layers of plastic, with instructions for storage and use inserted in the packaging, and deep frozen to −80 degrees Celsius.

The syringes and contents were sterilized by Cobalt-60 gamma irradiation at a dose of 25 kGy in the deep frozen state.

COB-05

The process used to prepare COB-10 was repeated but 5 cc of the material was loaded into the Becton-Dickinson 10 cc syringes.

COB-02

The process used to prepare COB-10 was repeated but 2 cc of the material was loaded into Becton-Dickinson 2 cc syringes.

COB-01

The process used to prepare COB-10 was repeated but 1 cc of the material was loaded into Becton-Dickinson 2 cc syringes.

CAB-02

The process used to prepare COB-10 was repeated but 40 cc of cancellous DFDBA was used and 2 cc of the material was loaded into Becton-Dickinson 2 cc syringes.

CAB-01

The process used to prepare COB-10 was repeated but 40 cc of cancellous DFDBA was used and 1 cc of the material was loaded into Becton-Dickinson 2 cc syringes.

CCAB-02

The process used to prepare COB-10 was repeated but 60 cc of cancellous DFDBA was used and 2 cc of the material was loaded into Becton-Dickinson 2 cc syringes.

CCAB-01

The process used to prepare COB-10 was repeated but 40 g of the cross-linked collagen was wet milled, 60 cc of cancellous DFDBA was used and 1 cc of the material was loaded into Becton-Dickinson 2 cc syringes.

CCAB-0.5

The process used to prepare COB-10 was repeated but 40 g of the cross-linked collagen was wet milled, 60 cc of cancellous DFDBA was used and 0.5 cc of the material was loaded into Becton-Dickinson 2 cc syringes.

CCAB-050 (5 cc)

The process used to prepare COB-10 was repeated but 40 g of the cross-linked collagen was wet milled, 60 cc of cancellous DFDBA was used and 5 cc of the material was loaded into Becton-Dickinson 10 cc syringes.

It is an advantage of the invention illustrated that the cross-linking method allows collagenous biomaterial to be cross-linked by gamma irradiation without any significant damage to the collagen molecule. Prior art methods used for the cross-linking of collagen by gamma irradiation have resulted in extensive damage to the collagen molecule and an accompanying change in the properties of the molecule. The invention therefore provides a method for the cross-linking of collagenous biomaterial without the use of chemical cross-linking agents and the accompanying cytotoxic dangers associated with chemically cross-linked collagen. It is not clear why the collagen in the gel is not damaged by gamma irradiation and the applicant believes that other components present in the collagenous biomaterial extract may play a role in reducing or preventing irradiation damage.

1-31. (canceled)

32. A method of producing a cross-linked collagenous biomaterial, the method including the step of providing a collagenous biomaterial and irradiating the collagenous biomaterial with gamma radiation at a dose of between about 20 and 160 kGy to produce the cross-linked collagenous biomaterial,

33. A method as claimed in claim 32, in which the collagenous biomaterial is provided in the form of a gel.

34. A method as claimed in claim 32, in which the collagenous biomaterial is irradiated at a dosage of between 70 and 90 kGy.

35. A method as claimed in claim 34, in which the collagenous biomaterial is irradiated at a dosage of about 80 kGy.

36. A method as claimed in claims 32, in which the collagenous biomaterial is produced by extracting bone powder.

37. A method as claimed in claim 32, in which the collagenous biomaterial is produced by extracting tendon.

38. A method as claimed in claim 36, in which the bone powder is defatted, dehydrated, milled human cortical bone powder.

39. A method as claimed in claim 36, in which the extraction is selected from an aqueous extraction and an aqueous acidic extraction.

40. A method as claimed in claim 32, in which the collagenous biomaterial is irradiated with a cobalt 60 source.

41. A cross-linked collagenous biomaterial produced by a method as claimed in claim 32.

42. A tissue-regenerating composition which includes
a cross-linked collagenous biomaterial produced by a method as claimed in in claim 32, and
at least one component selected from processed bone, insoluble collagenous bone matrix, bone growth inducing factors, cortical bone particles, cancellous bone particles, hydroxyapatites, ceramic powders, demineralised bone particles and mixtures of any two or more thereof.

43. A composition as claimed in claim 42, in which the bone growth inducing factors are selected from bone morphogenetic proteins, transforming growth factor beta and combinations thereof.

44. A tissue-regenerating device which includes a tissue-regenerating composition as claimed in claim 42 and administration means for administering the composition to a treatment site.

45. A tissue-regenerating device as claimed in claim 44, in which the administration means is a syringe.

46. A tissue-regenerating device as claimed in claim 44, in which the administration means is in the form of a membrane for use in guided tissue regeneration.

47. A tissue-regenerating device as claimed in claim 44, in which the administration means is in the form of a haemostatic sponge for use in stopping haemorrhagic trauma of internal organs.

48. A tissue-regenerating device as claimed in claim 44, in which the administration means is in sheet form for use in covering skin burns.

49. A method of regenerating tissue, the method including the step of administering to a person or animal in need of treatment an effective amount of a substance or composition which includes a cross-linked collagenous biomaterial prepared by a method as claimed in claim 32.

50. A method as claimed in claim 49, in which the substance or composition includes at least one component selected from processed bone, insoluble collagenous bone matrix, bone growth inducing factors, cortical bone particles, cancellous bone particles, hydroxyapatites, ceramic powders, demineralised bone particles and mixtures of any two or more thereof.
